(19) United States
(12) Patent Application Publication
(10) Pub. No.: US 2007/0077293 A1
(43) Pub. Date: Apr. 5, 2007

(54) Film-forming composition suitable for hard capsules and method for preparing the same

(75) Inventors: Hyun-Jin Park, Seoul (KR); Ho-Jae Bae, Gyeonggi-do (KR)

Correspondence Address: BUCHANAN, INGERSOLL & ROONEY PC, POST OFFICE BOX 1404, ALEXANDRIA, VA, 22313-1404, US

(73) Assignees: Young-Soo CHA, Seoul (KR); Hyun-Jin PARK, Seoul (KR)

(21) Appl. No.: 10/580,853

(22) PCT Filed: Dec. 3, 2003
(86) PCT No.: PCT/KR03/02637

§ 371 Date: May 26, 2006

(30) Foreign Application Priority Data
Nov. 26, 2003 (KR) 10-2003-0084391

(51) Int. Cl. A61K 9/48

(52) U.S. Cl. 424/451

(57) ABSTRACT

The present invention relates to a film-forming composition suitable for hard capsules, and a preparation method thereof. More particularly, the invention relates to a film-forming composition for hard capsules, comprising 7-12% by weight of starch, 1-6% by weight of a plasticizer, 0.7-3% by weight of a gelling agent, and 79-91.3% by weight of water, and a preparation method thereof. Also, the invention relates to films and hard capsules comprising the composition. The inventive composition, and the hard capsules and films comprising the same, will be useful in various industrial fields, including pharmaceutical field and food field.

### TECHNICAL FIELD

The present invention relates to a film-forming composition suitable for hard capsules, and a preparation method thereof, and more particularly to a film-forming composition comprising starch, and a preparation method thereof.

### BACKGROUND ART

Generally, many medicines and dietary supplements (health supplementary food) are prepared in the form of capsules. Capsules include soft capsules and hard capsules. The soft capsules have a surface which is soft and consist of one piece, whereas the hard capsules have a surface which is harder than that of the soft capsules and consist of two pieces. The soft capsules are mainly filled with suspension, paste or emulsion materials as contents while the hard capsules are mainly filled with powders or granules. Most medicines are prepared as hard capsules. Moreover, more than 70% of herb supplements among dietary supplements in the United States are prepared as hard capsules. Also, in the case of dietary supplements and special nutritional foods manufactured and distributed in Korea, many products are manufactured in the form of hard capsules.

Meanwhile, gelatin is a derived protein which is obtained by treating collagen derived from animal skins or bones with acids or alkalis and then extracting the treated collagen with hot water. It is known that gelatin is made of repeated bonds of proline or hydroxyproline, glycerin and other amino acids, which are present in the ratio of 2:3:4 when the gelatin is derived from domestic animal skins. Gelatin is used as a base material for capsules, as it has excellent film-forming capability and physical properties. U.S. Pat. No. 5,456,746 discloses a hard film composition for forming gelatin capsules, and U.S. Pat. No. 5,419,916 discloses a gelatin coating composition for preparing hard capsules. However, gelatin is disadvantageous in that it is expensive so that many costs are needed in the preparation of hard capsules using the same. Furthermore, on the ground that gelatin is an animal component, vegetarians and many religious people avoid the use of capsules made with gelatin. Particularly due to the recent appearance of bovine spongiform encephalopathy, the risk of animal protein products is seriously suggested, so that the use of gelatin is limited. Because of such disadvantages of gelatin, there is now a need for the development of capsules using plant-based materials in order to replace gelatin.

On this, a capsule using hydroxypropyl methyl cellulose (HPMC), a plant-based component to replace gelatin, was developed; European Patent No. 714656 discloses a film composition comprising HPMC, carrageenan, and calcium or potassium ions. Also capsules using starch as a substitute for gelatin was developed. U.S. Pat. No. 5,451,763 discloses a method of preparing a film by pouring a solution of starch and pectin in water on a film-forming plate and evaporating water. However, this method has a problem in that a starch having an amylose amount of 40% must be used, resulting in a reduction in the oxygen barrier properties of the film. Moreover, U.S. Pat. No. 6,582,727 discloses a film composition suitable for soft capsules, which comprises modified starch, ι-carrageenan, a plasticizer, and the like. In the prior art, capsules prepared with starch are soft capsules, and there is no disclosure of either a composition for hard starch capsules or a preparation method thereof.

### DISCLOSURE OF THE INVENTION

Therefore, the present inventors have continued to study to prepare plant-based capsules suitable for hard capsules and consequently discovered the optimum amounts of starch, a plasticizer and a gelling agent, thereby completing the present invention.

Accordingly, it is an object of the present invention to provide a film-forming composition suitable for hard capsules.

Another object of the present invention is to provide a method for preparing said composition.

Still another object of the present invention is to provide a film and a hard capsule comprising said composition.

To achieve the above objects, in one aspect, the present invention provides a film-forming composition for hard capsules, comprising 7-12% by weight of starch, 1-6% by weight of a plasticizer, 0.7-3% by weight of a gelling agent, and 79-91.3% by weight of water.

In another aspect, the present invention provides a method for preparing a film-forming composition for hard capsules, the method comprising the steps of:

(a) adding 1-6% by weight of a plasticizer based on the total weight of the composition to 79-91.3% by weight of water based on the total weight of the composition and stirring the solution at 25-60° C.;

(b) adding 0.7-3% by weight of a gelling agent based on the total weight of the composition to the solution prepared in the step (a), and stirring the gelling agent-comprising solution at 50±5° C.; and

(c) adding 7-12% by weight of starch based on the total weight of the composition to the solution prepared in the step (b), and stirring the starch-comprising solution while gradually elevating the temperature of the solution to 90±5° C.

In still another aspect, the present invention provides a film-forming composition for hard capsules, comprising 5-11% by weight of waterchestnut starch or mungbean starch, 1-3% by weight of a plasticizer, and 88-94% by weight of water.

In yet another aspect, the present invention provides a method for preparing a film-forming composition for hard capsules, the method comprising the steps of:

(a) adding 1-3% by weight of a plasticizer based on the total weight of the composition to 88-94% by weight of water based on the total weight of the composition, and stirring the solution at 25-60° C.; and

(b) adding 5-11% by weight of waterchestnut starch or mungbean starch based on the total weight of the composition to the solution prepared in the step (a), and stirring the starch-comprising solution while gradually elevating the temperature of the solution to 90±5° C.

In further another aspect, the present invention provides a film and a hard capsule comprising said composition.

### BRIEF DESCRIPTION OF THE DRAWINGS

FIG. 1 shows the configuration of a stirring impeller preferred for use in preparing a film-forming composition according to the present invention.

FIG. 2 is a photograph showing each of hard capsules prepared in the case of a suitable starch amount (A) and the cases of starch amounts of more than 70 g (B: no removal of bubbles; and C: tailing occurred).

FIG. 3 illustrates photographs showing the shape of hard capsules (A) and the shape of films during a drying process (B), in the case of a plasticizer amount of less than 3 g.

FIG. 4 is a photograph showing the shape of hard capsules prepared in the case of a gelling agent amount of less than 3 g.

FIG. 5 is a photograph showing the shape of hard capsules prepared in the present invention. Starting from the left side in FIG. 5, the hard capsules are an HPMC capsule (Su-Heung Capsule Co., Ltd, Korea), an HPMC capsule (Capsugel, France), and a potato starch capsule, mungbean starch capsule and waterchestnut starch capsule of the present invention.

FIG. 6 shows the shape of mungbean starch capsules prepared with no addition of a gelling agent.

FIG. 7 shows the shape of chestnut starch capsules prepared with no addition of a gelling agent.

FIG. 8 is a graph showing measurement results for the paste viscosity of potato starch.

FIG. 9 is a graph showing measurement results for the paste viscosity of mungbean starch.

FIG. 10 is a graph showing measurement results for the paste viscosity of waterchestnut starch.

### BEST MODE FOR CARRYING OUT THE INVENTION

Hereinafter, the present invention will be described in detail.

The present invention provides a composition suitable for hard capsules using starch (a natural plant-based component) as a base material. Specifically, the present invention provides a film-forming composition for hard capsules, comprising a small amount of starch, a plasticizer, a gelling agent and water.

The composition according to the present invention comprises starch in an amount of 7-12% by weight based on the total weight of the composition. The amount of starch has the greatest effect on the preparation of capsules. If the starch amount is less than 7% by weight, there will be a problem in that the composition is too sloppy so that it is not prepared into capsules at all, or it is not prepared in a thickness range suitable for hard capsules (post-drying thickness: 0.1-0.15 mm). On the other hand, if the starch amount exceeds 12% by weight, bubbles in a film will not be removed, and upon dipping of capsules, tailing at the end of pins will occur, thereby making the production of high-quality capsules difficult. Starches which can be used in the present invention include potato starch, sweet potato starch, waterchestnut starch, mungbean starch, coin starch, wheat starch, rice starch, glutinous rice starch, amaranth starch, barley starch, tapioca starch and the like. Preferably, one selected from the group consisting of potato starch, waterchestnut starch and mungbean starch may be used. The preferred starch which can be used in the present invention has an amylose content of 10-30% by weight (see Table 12). Moreover, the preferred paste viscosity of a starch which can be used in the present invention is as described in Table 13.

Furthermore, the film-forming composition according to the present invention comprises a plasticizer in an amount of 1-6% by weight based on the total weight of composition. If the amount of the plasticizer is less than 1% by weight, capsules will be broken when releasing the capsules from dipping pins, or the prepared capsules or films will not be flexible so that the capsules will be collapsed just even when applying only a little force to the capsules. On the other hand, if the plasticizer amount exceeds 6% by weight, the prepared capsules will become too soft, so that they cannot be maintained in the form of hard capsules. Also if the plasticizer is added in an excessively large amount, the oxygen permeability of capsules will be increased. The amount of addition of the plasticizer is preferably controlled depending on the amount of addition of solids (starch and a gelling agent) used. Preferably, the plasticizer is added in amount of about 25% of the solids. In the present invention, any plasticizer may be used without limitations insofar as it is a general plasticizer which is used in the preparation of capsules. Preferably, as the plasticizer, it is preferable to use either any one selected from the group consisting of glycerol, sorbitol, polyethylene glycerol (PEG), mannitol and maltitol, or a mixture thereof. More preferably, glycerin or glycerol may be used.

The film-forming composition according to the present invention comprises a gelling agent in an amount of 0.7-3% by weight based on the total weight of the composition. The amount of the gelling agent has a close connection with when separating the dried capsules from dipping pins. If the amount of the gelling agent is less than 0.7% by weight, the strength of capsules will be weak, so that when separating the capsules from dipping pins, the capsules will be deformed in the separation direction. Also, when dipping the pins into a film-forming solution, the solution will not securely adhere to the pin surface. On the other hand, if the gelling agent is used in a too large amount, capsules with uniform thickness cannot be prepared. In the present invention, any gelling agent may be used without limitations insofar as it is a general gelling agent which is used in the preparation of hard capsules. It is preferable to use either any one selected from the group consisting of carrageenan, agar gum, pectin, alginate, gum arabic, xanthan gum and gellan, or a mixture thereof. More preferably, κ-carrageenan or a mixture of κ-carrageenan and ι-carrageenan may be used.

The film-forming composition according to the present invention may additionally comprise a pigment. The pigment enables capsules to show a beautiful shape and color and to be distinguished from others. Any pigment may be used in the present invention without limitations insofar as it is a general pigment which is used in the preparation of capsules. Preferably, edible pigment Blue No. 1, Red No. 3, Red No. 40, Yellow No. 4 or Yellow No. 5 which is listed in the Korean Food Additive Code may be used. Since the color or nature of contents filled in capsules influences the color of a capsule film, it is preferable to determine a pigment in view of the color of the contents. In addition, the composition according to the present invention may additionally comprise an opacifier such as titanium oxide, or a preservative such as para-benzoic acid, etc. The inventive composition preferably has a viscosity of 600-2250 cps.

The inventive composition is preferably prepared by performing the mixing of the components in the following order: water, the plasticizer, the gelling agent, and starch. If starch is first added to water, the viscosity of the solution will be increased, so that when the plasticizer and the gelling agent are added, they will not be well dispersed in the solution. Particularly, if the gelling agent is added following the addition of the starch, masses of the gelling agent will drift in the solution even when stirring is performed over an extended period of time. For this reason, the mixing of the components is preferably performed in the above-described order. Hereinafter, the method for preparing the inventive composition will be described in more detail.

First, 1-6% by weight of the plasticizer based on the total weight of the film-forming composition is added to 79-91.3% by weight of water based on the total weight of the composition, and the solution is stirred at 25-60° C., and preferably 50±5° C. The stirring is preferably carried out for 15 minutes to 1 hour, and more particularly 30 minutes.

Then, to the mixture solution of water and the plasticizer, the gelling agent is added in an amount of 0.7-3% by weight based on the total weight of the film-forming composition. At this time, the temperature of the solution is preferably maintained at 50±5° C. Generally, the dissolution of the gelling agent is faster at higher temperature. Through various experiments, the present inventors have found that the most suitable temperature at which the gelling agent can be dissolved without gelatinizing the starch is 50±5° C. The stirring time of the solution after the addition of the gelling agent is preferably 20 minutes to 1 hour, and more particularly 30 minute.

To the mixture solution of water, the plasticizer and the gelling agent, which has been prepared as described above, 7-12% by weight of starch is finally added and stirred. Similarly to the step of adding the gelling agent, the temperature of the solution upon the addition of the starch is preferably 50±5° C. The gelatinization temperature of general starch is 70-100° C. If the temperature of water upon the addition of the starch to water is in the range of gelatinization temperature of the starch, the starch will be gelatinized in the order of starch portions which will be in contact with water, so that any portion of the starch will be gelatinized and any other portion of the starch will remain as powder. Accordingly, the gelatinization of the whole starch will not be achieved, so that films or capsules cannot be prepared.

Even when the temperature of water exceeds only 60° C., the partial gelatinization of the starch will occur.

After the addition of the starch, as the starch is sufficiently dispersed in the solution, the solution is stirred while gradually elevating the temperature of the solution to 90±5° C. As the temperature of the solution reaches 90±5° C., the solution is further stirred for 30-40 minutes. If the solution is further stirred for longer than 40 minutes, the starch solution will become undesirably loose.

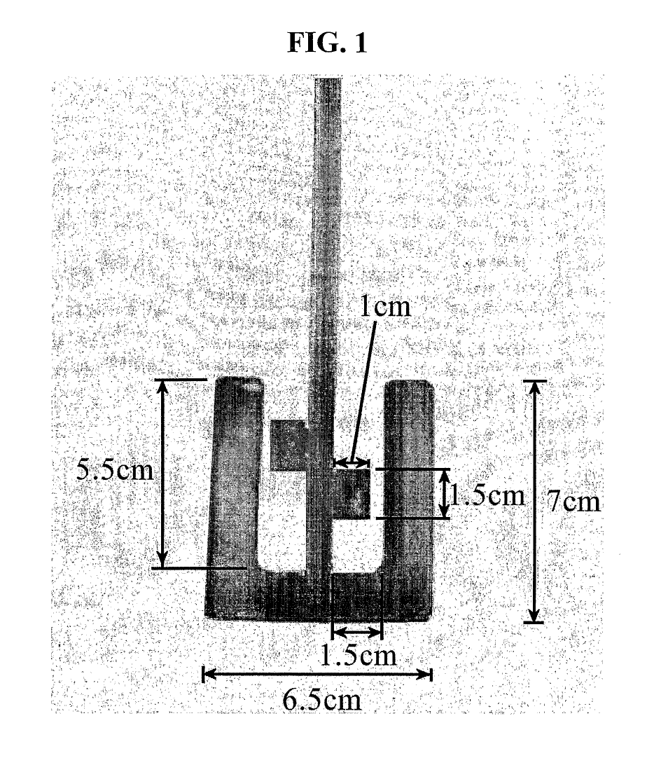

The temperature control of the solution upon stirring is preferably performed by the use of a water bath whose temperature can be controlled. Also, the stirring is carried out by means of a mechanical stirrer at a speed of 150-400 rpm, and preferably 300 rpm. For uniform mixing of each of the components, an impeller in the mechanical stirrer is preferably an anchor type as shown in FIG. 1. The solution prepared as described above has a viscosity of 600-2250 cps suitable for the preparation of hard capsules.

During studies to prepare hard capsules using starch, the present inventors have found that, in the case of use of mungbean starch or waterchestnut starch, hard capsules can be prepared even when the gelling agent is not added. Accordingly, the present invention provides a film-forming composition comprising 5-11% by weight of mungbean starch or waterchestnut starch, 1-3% by weight of the plasticizer and 88-94% by weight of water. This composition may be prepared in the same manner as in the above-described preparation method except that the gelling agent is not added. A specific preparation method of this composition comprises the steps of:

(a) adding 1-3% by weight of the plasticizer based on the total weight of the composition to 88-94% by weight of water based on the total weight of the composition, and stirring the solution at 25-60° C.; and

(b) adding 5-11% by weight of waterchestnut starch or mungbean starch based on the total weight of the composition to the solution prepared in the step (a), and stirring the starch-comprising solution while gradually elevating the temperature of the solution to 90±5° C.

Furthermore, the present invention provides a hard capsule and a film, comprising the inventive film-forming composition. The hard capsule and film may be prepared by any conventional method known in the art. For the hard capsule, an immersion coating process using dipping pins, which is generally used in the hard capsule manufacturing industry, may preferably be used. For the film, a film casting machine may preferably be used. The preparation of the hard capsule and the film is preferably performed while maintaining the temperature of the inventive composition at 70-80° C. If the temperature of the composition is lowered below 70° C., the rapid gelling of the composition will occur, thereby making it impossible to prepare capsules and films with uniform thickness. The physical properties of films prepared with the inventive composition are given in Tables 7 and 8 below. Particularly, the films prepared with the inventive composition are very excellent in oxygen barrier properties. Also, the hard capsules prepared with the inventive composition have a water content of less than 5% (see Table 9).

### EXAMPLES

Hereinafter, the present invention will be described in detail by examples. It is to be understood, however, that these examples are given for illustrative purpose only and are not construed to limit the scope of the present invention. Unless stated otherwise, the unit (g) of each component described herein is expressed as the dry basis weight of solids.

### Experimental Example 1

### Determination of Starch Amount Suitable for Hard Capsules

In order to determine the amount of starch suitable for the preparation of hard capsules, the present inventors have prepared hard capsules and films which comprise starch in various amounts. Potato starch was used as starch, glycerol as a plasticizer, and κ-carrageenan as a gelling agent. First, glycerol (Junsei, Tokyo, Japan) was added to deionized water, and the solution was mixed with a mechanical stirrer equipped with an anchor-type impeller as shown in FIG. 1, at a speed of 300 rpm and 50±5° C. for 30 minutes. The temperature of the solution was controlled by a water bath whose temperature can be controlled. Then, to the mixture solution of glycerol and deionized water, κ-carrageenan (MSC Co., LTD, Soju, Ungsang, Yangsan, Kyeongnam, Korea) was added, and the solution was mixed with the mechanical stirrer under the same conditions as described above. The potato starch (AVEBE B.A., Postbus 15, 9640 AA Veendam, The Netherlands) was then added in various amounts to the κ-carrageenan-comprising solution. Next, the solution was stirred while gradually elevating the temperature of the water bath to 90±5° C. After the temperature of the water bath reached 90±5° C., the solution was further stirred for 40 minutes.

TABLE 1
Tests for determination of starch amount suitable for the preparation of hard capsules

| | Amount of addition (g) | | | | | | | | | | | | | |
|---|---|---|---|---|---|---|---|---|---|---|---|---|---|---|
| Potato starch | 5 | 10 | 11 | 12 | 13 | 14 | 15 | 20 | 30 | 40 | 50 | 60 | 70 | 80 |
| Glycerin | 10 | 10 | 10 | 10 | 10 | 10 | 10 | 10 | 10 | 10 | 10 | 10 | 10 | 10 |
| κ-carrageenan | 1 | 1 | 1 | 1 | 1 | 1 | 1 | 1 | 1 | 1 | 1 | 1 | 1 | 1 |
| Water | 450 | 450 | 450 | 450 | 450 | 450 | 450 | 450 | 450 | 450 | 450 | 450 | 450 | 450 |
| Total amount | 466 | 471 | 472 | 473 | 474 | 475 | 476 | 481 | 491 | 501 | 511 | 521 | 531 | 541 |

Hard capsules and films were prepared with the film-forming solution as described above. First, hard capsule dipping pins (Su-Heung Capsule Co., Ltd, Korea) were immersed in the film-forming solution while maintaining the temperature of the solution at 70-80° C. At this time, the dipping pins were immersed perpendicularly in the film-forming solution up to about 4/5 of their length, and drawn out slowly at uniform speed after about 5 seconds. Next, the solution on the dipping pins was dried for 12 hours in a constant temperature and humidity chamber controlled to 50% RH and 25° C. The prepared hard capsules were released from the dipping pins.

Meanwhile, films were prepared by means of a film casting machine (PI-1210 Filmcoater, Tester Sangyo Co., LTD, Tokyo, Japan). For this purpose, the film-forming solution was cast on a Teflon-coated glass plate (25 cm×35 cm). At this time, the speed of a film applicator was controlled to 10-15 mm/sec. Then, the film-forming solution on the glass plate was dried for 12 hours in a constant temperature and humidity chamber controlled to 50% RH and 25° C. The prepared films were released from the glass plate.

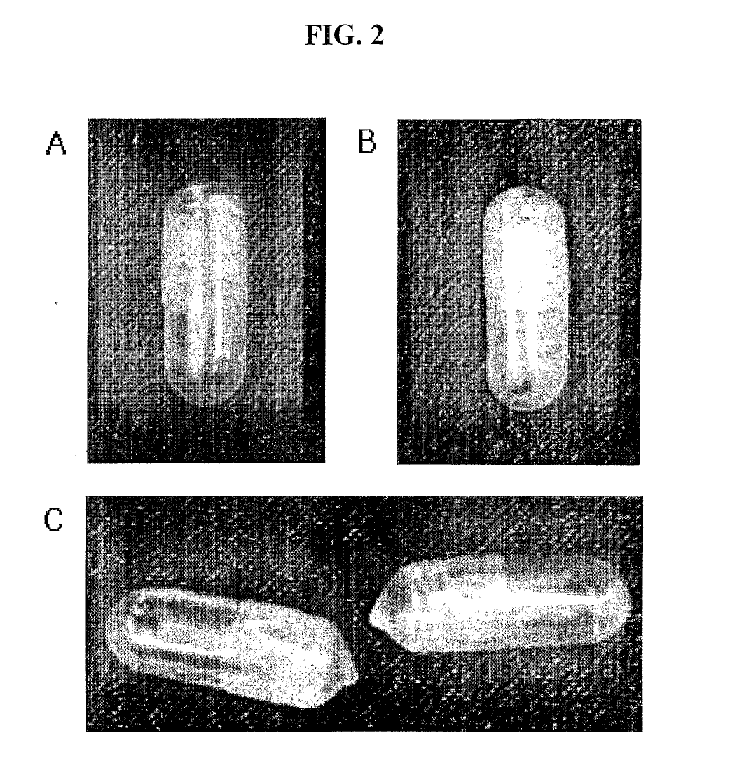

The capsules and films prepared as described above were examined for their shapes and physical properties, and the results showed that, in the case of a starch amount of more than 70 g, there was a problem in that bubbles on the surfaces of the capsules and the films were not removed, or tailing at the end of the pins occurred during the dipping process of the hard capsules (see FIGS. 2B and 2C). On the other hand, in the case of a starch amount of less than 30 g, bubbles on the films were removed but there was a problem in that the film-forming solution upon the preparation of the capsules became too sloppy, so that a sufficient amount of the solution did not adhere to the pins, and thus, the thickness of the capsules obtained after drying became too thin (less than 0.1 mm), thereby making it difficult to use the capsules as products.

### Experimental Example 2

### Determination of Plasticizer Amount Suitable for Hard Capsules

In order to determine the amount of the plasticizer suitable for the preparation of hard capsules, the present inventors have prepared the hard capsules and films comprising the plasticizer in various amounts as described in Table 2 below, in the same manner as in Experimental Example 1. Potato starch was used as the starch, glycerol as the plasticizer, and κ-carrageenan as the gelling agent. The amount of the starch was 9% by weight which is within the optimum amount range determined in Experimental Example 1.

TABLE 2
Test for determination of plasticizer amount suitable for hard capsule

| | Amount of addition (g) | | | | | | | | | | | | | | | |
|---|---|---|---|---|---|---|---|---|---|---|---|---|---|---|---|---|
| Potato starch | 45 | 45 | 45 | 45 | 45 | 45 | 45 | 45 | 45 | 45 | 45 | 45 | 45 | 45 | 45 | 45 |
| Glycerol | 0 | 3 | 6 | 9 | 12 | 15 | 18 | 21 | 24 | 27 | 30 | 33 | 36 | 40 | 50 | 60 |
| κ-carrageenan | 1 | 1 | 1 | 1 | 1 | 1 | 1 | 1 | 1 | 1 | 1 | 1 | 1 | 1 | 1 | 1 |
| Water | 450 | 450 | 450 | 450 | 450 | 450 | 450 | 450 | 450 | 450 | 450 | 450 | 450 | 450 | 450 | 450 |
| Total weight | 496 | 499 | 502 | 505 | 508 | 511 | 514 | 517 | 520 | 523 | 526 | 529 | 531 | 536 | 546 | 556 |

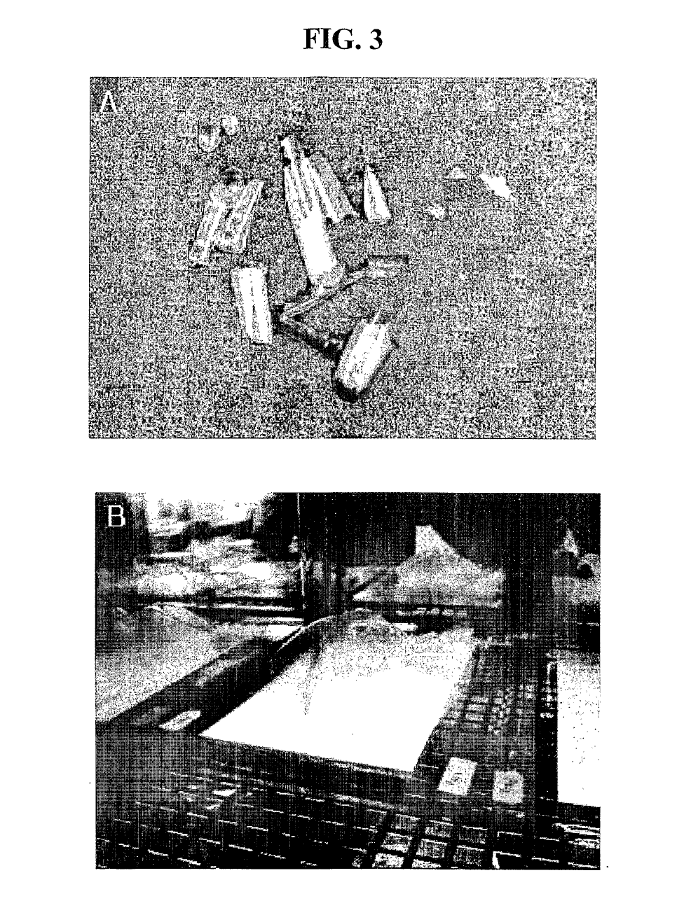

The shape and physical properties of the prepared hard capsules were examined, and the results showed that, in the case of a plasticizer amount of less than 3 g, the capsules were broken upon separation from the dipping pins, or the films were shrunk during drying process (see FIG. 3). In the case of no addition of the plasticizer, capsules were not prepared at all. On the other hand, in the case of a plasticizer amount of more than 33 g, there was a problem in that the capsules became excessively soft, so that the configuration of the capsules was distorted. When an excessive amount of the plasticizer was added, the oxygen permeability of the prepared film would be increased (data not shown). Particularly, it was most suitable for the preparation of hard capsules to add the plasticizer at about 25% by weight based on solids (starch and gelling agent).

### Experimental Example 3

### Determination of Gelling Agent Amount Suitable for Jard Capsules

In order to determine the amount of the gelling agent suitable for the preparation of hard capsules, the present inventors have prepared the hard capsules and films comprising the gelling agent in various amounts as shown in Table 3 below, in the same manner as in Experimental Example 1. Potato starch was used as the starch, glycerol as the plasticizer, and κ-carrageenan as the gelling agent. The amounts of the starch and the plasticizer were set to about 9% by weight and about 2-3% by weight, respectively, which are in the optimum amount ranges determined in Experimental Examples 1.

TABLE 3
Determination of amount of gelling agent suitable for hard capsules

| | Amount of addition (g) | | | | | | | | |
|---|---|---|---|---|---|---|---|---|---|
| Mungbean starch | 45 | 45 | 45 | 45 | 45 | 45 | 45 | 45 | 45 |
| Glycerin | 11.25 | 11.5 | 11.75 | 12 | 12.25 | 12.5 | 12.75 | 13 | 13.25 |
| κ-carrageenan | 0 | 1 | 2 | 3 | 4 | 5 | 6 | 7 | 8 |
| Water | 450 | 450 | 450 | 450 | 450 | 450 | 450 | 450 | 450 |
| Total amount | 506.25 | 507.5 | 508.75 | 510 | 511.25 | 512.5 | 513.75 | 515 | 516.25 |
| Mungbean starch | 45 | 45 | 45 | 45 | 45 | 45 | 45 | 45 | 45 |
| Glycerin | 13.5 | 13.75 | 14 | 14.25 | 14.5 | 14.75 | 15 | 16.25 | 17.5 |
| κ-carrageenan | 9 | 10 | 11 | 12 | 13 | 14 | 15 | 20 | 25 |
| Water | 450 | 450 | 450 | 450 | 450 | 450 | 450 | 450 | 450 |
| Total amount | 517.25 | 518.75 | 520 | 520.25 | 522.5 | 523.75 | 525 | 531.25 | 537.5 |

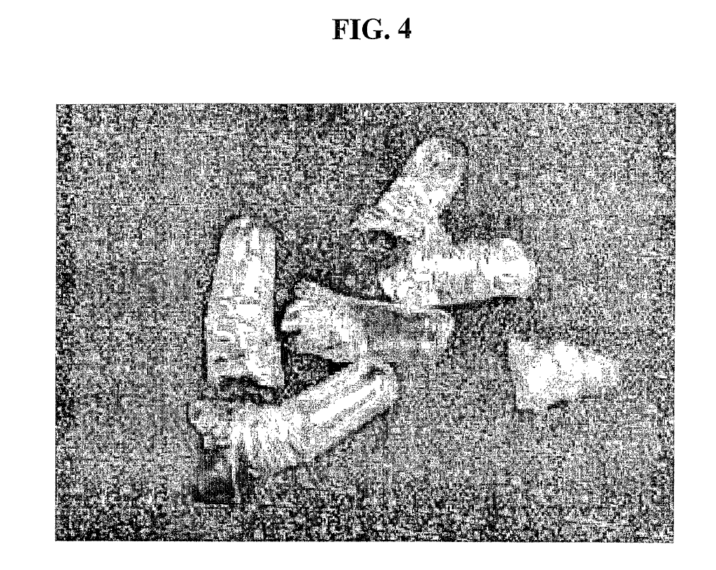

The shape and physical properties of the prepared hard capsules and films were examined, and the results that, in the case of a gelling agent amount of less than 3 g, there was a problem in that the strength of the prepared capsules was weak, so that the capsules upon separation from the dipping pins were crumpled (see FIG. 4). In the case of a gelling agent amount of more than 20 g, there were problems in that the thickness of the films became non-uniform, and the film-forming solution did not adhere uniformly to the surface of the pins so that suitable capsules were not prepared.

### Example 1

### Preparation of Film and Hard Capsule Using Potato Starch

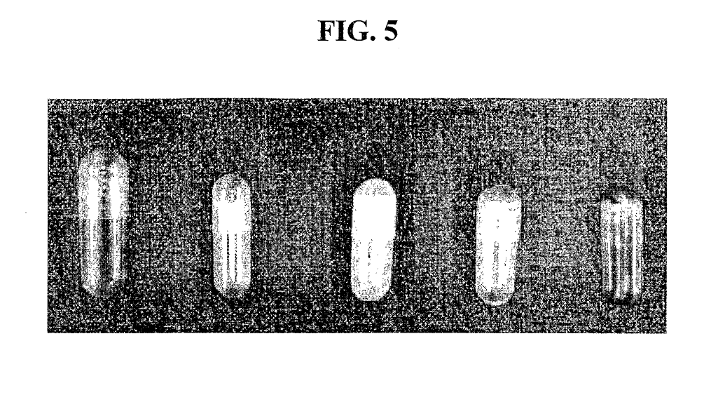

Using potato starch (AVEBE B.A., Postbus 15, 9640 AA Veendam, The Netherlands), a film and a hard capsule were prepared in the same manner as in Experimental Example 1. A photograph of the prepared hard capsule with potato starch is shown in FIG. 5. The potato starch-comprising composition is composed as Table 4 below.

TABLE 4
Film-forming composition comprising potato starch

| | Amount of addition (g) |
|---|---|
| Potato starch | 45.0 |
| κ-carrageenan | 6.6 |
| Glycerin | 11.25 |
| Water | 450 |
| Total amount | 512.85 |

### Example 2

### Preparation of Film and Hard Capsule Using Mungbean Starch

Using mungbean starch (Rhee Brothers, Columbia, Md., US), a film and a hard capsule were prepared in the same manner as in Experimental Example 1. A photograph of the prepared hard capsule with mungbean starch is shown in FIG. 5. The mungbean starch-comprising composition is composed as Table 5 below.

TABLE 5
Film-forming composition comprising mungbean starch

| | Amount of addition (g) |
|---|---|
| Mungbean starch | 50.0 |
| κ-carrageenan | 3.3 |
| ι-carrageenan | 3.3 |
| Glycerin | 11.0 |
| Water | 450 |
| Total amount | 517.6 |

### Example 3

### Preparation of Film and Hard Capsule Using Waterchestnut Starch

Using waterchestnut starch (Pan Tang Brand, Guangzhou, China), a film and a hard capsule were prepared in the same manner as in Experimental Example 1. A photograph of the prepared hard capsule with waterchestnut starch is shown in FIG. 5. The waterchestnut starch-comprising composition is composed as Table 6 below.

TABLE 6
Film-forming composition comprising waterchestnut starch

| | Amount of addition (g) |
|---|---|
| Waterchestnut starch | 45.0 |
| κ-carrageenan | 3.3 |
| ι-carrageenan | 3.3 |
| Glycerin | 11.25 |
| Water | 450 |
| Total amount | 512.85 |

### Example 4

### Examination of Physical Properties of Starch Films Prepared in Present Invention

Each of the starch films prepared in Examples 1-3 was measured for tensile strength, elongation and oxygen permeability.

(4-1) Measurement of Tensile Strength and Elongation

The tensile strength and elongation of the films were measured according to the ASTM standard method D 882-88 (ASTM, 1989). The measurement results are shown in Table 7 below.

TABLE 7
Measurement results of tensile strength and elongation

| | Thickness of film | Mechanical properties | |
|---|---|---|---|
| | after drying | Tensile strength (MPa) | Elongation (%) |
| Example 1 | 0.107 ± 0.000690 | 34.98 ± 1.40 | 4.07 ± 0.36 |
| Example 2 | 0.332 ± 0.0787 | 12.17 ± 2.71 | 60.08 ± 8.69 |
| Example 3 | 0.233 ± 0.02132 | 13.47 ± 1.07 | 43.32 ± 7.09 |

As shown Table 7, the potato starch film prepared in Example 1 showed the highest tensile strength, and the mungbean starch film prepared in Example 2 showed the highest elongation.

(4-2) Measurement of Oxygen Permeability

The oxygen permeability of the films was measured with an OX-Tran 2/60 O₂ transmission tester (Mocon Modem Control, Inc., Minneapolis, Minn.) according to the ASTM standard method D 3985-81 (ASTM, 1989b). As a control group, a commercially available hard gelatin capsule (Su-Heung Capsule Co., Ltd) and hard HPMC capsule (Su-Heung Capsule Co., Ltd) were used. The measurement results are shown in Table 8 below.

TABLE 8
Measurement results of oxygen permeability

| | Oxygen permeability (cc/m² · day) |
|---|---|
| Example 1 | 2.32 |
| Example 2 | 2.27 |
| Example 3 | 1.66 |
| Hard gelatin capsule | 1.15 |
| Hard HPMC capsule | 260 |

As shown in Table 8, the oxygen permeability of the films prepared in the present invention was slightly higher than that of the hard gelatin capsule, but remarkably lower than that of the hard HPMC capsule. This suggests that the hard starch capsule prepared according to the present invention is very excellent in oxygen barrier properties as compared to the hard HPMC capsule, a prior plant-based hard capsule.

### Example 5

### Measurement of Water Content of Hard Starch Capsules Prepared in Present Invention

The hard capsules prepared in Examples 1 to 3 were examined for water content. The water content was obtained by measuring pre-drying weight and post-drying weight and calculating loss-on-drying (%) from the measured values. The measurement results are given in Table 9 below.

TABLE 9
Measurement results of water content

| | Pre-drying | Post-drying | |
|---|---|---|---|
| | weight (g) | weight (g) | Loss-on-drying (%) |
| Example 1 | 2.702 | 2.569 | 4.92 |
| Example 2 | 2.990 | 2.847 | 4.78 |
| Example 3 | 3.068 | 2.949 | 3.88 |

As shown in Table 9, the hard starch capsules prepared in the present invention all showed water content of less than 5%. These results demonstrated that the hard starch capsules prepared in the present invention had stability against aging, in view of a report that if the water content of pre-gelatinized products is limited to less than 15%, the aging of the starch products will be inhibited (Kim Dong-Hoon, Food Chemistry, Tarngudang, 2001).

### Example 6

### Preparation of Hard Starch Capsule with No Addition of Gelling Agent

(6-1) Preparation of Mungbean Starch-hard Capsule

Hard capsules and films were prepared with compositions described in Table 10 below in the same manner as in Experimental Example 1 except that the step of adding the gelling agent was not performed. The amount of the plasticizer was 0.2-3% by weight based on the amount of starch.

TABLE 10
Preparation of mungbean starch-hard capsules with no addition of gelling agent

| | Amount of addition (g) | | | | | | | | | | | | |
|---|---|---|---|---|---|---|---|---|---|---|---|---|---|
| Mungbean starch | 5 | 10 | 15 | 20 | 25 | 30 | 35 | 40 | 45 | 50 | 55 | 60 | 65 |
| Glycerin | 1.25 | 2.5 | 3.75 | 5 | 6.25 | 7.5 | 8.75 | 10 | 11.25 | 12.5 | 13.75 | 15 | 16.25 |
| Water | 450 | 450 | 450 | 450 | 450 | 450 | 450 | 450 | 450 | 450 | 450 | 450 | 450 |
| Total weight | 456.25 | 462.5 | 468.75 | 475 | 481.25 | 487.5 | 493.75 | 500 | 506.25 | 512.5 | 518.75 | 525 | 531.25 |

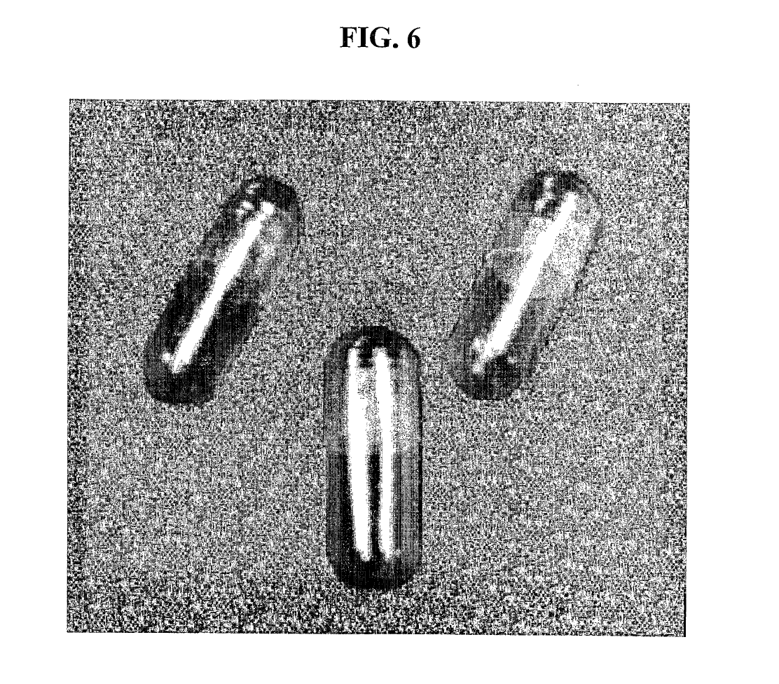

As shown in FIG. 6, the results showed that, if the amount of mungbean starch was 25-55 g, it would be prepared into hard capsules even when the gelling agent was not added. However, if the amount of the starch was less than 20 g, the thickness of the film after drying would be less than 0.1 mm. On the other hand, if the amount of the starch was more than 60 g, there would be a problem in that the solution was too viscous, so that tailing upon the preparation of capsules occurred, or bubbles were not removed, or the thickness of the film was not uniform.

(6-2) Preparation of Waterchestnut Starch-hard Capsules

Hard capsules and films were prepared with compositions described in Table 11 below in the same maimer as in Experimental Example 1 except that the step of adding the gelling agent was not performed.

TABLE 11
Preparation of waterchestnut starch-hard capsules with no addition of gelling agent

| | Amount of addition (g) | | | | | | | | | | | | |
|---|---|---|---|---|---|---|---|---|---|---|---|---|---|
| Mungbean starch | 5 | 10 | 15 | 20 | 25 | 30 | 35 | 40 | 45 | 50 | 55 | 60 | 65 |
| Glycerin | 1.25 | 2.5 | 3.75 | 5 | 6.25 | 7.5 | 8.75 | 10 | 11.25 | 12.5 | 13.75 | 15 | 16.25 |
| Water | 450 | 450 | 450 | 450 | 450 | 450 | 450 | 450 | 450 | 450 | 450 | 450 | 450 |
| Total amount | 456.25 | 462.5 | 468.75 | 475 | 481.25 | 487.5 | 493.75 | 500 | 506.25 | 512.5 | 518.75 | 525 | 531.25 |

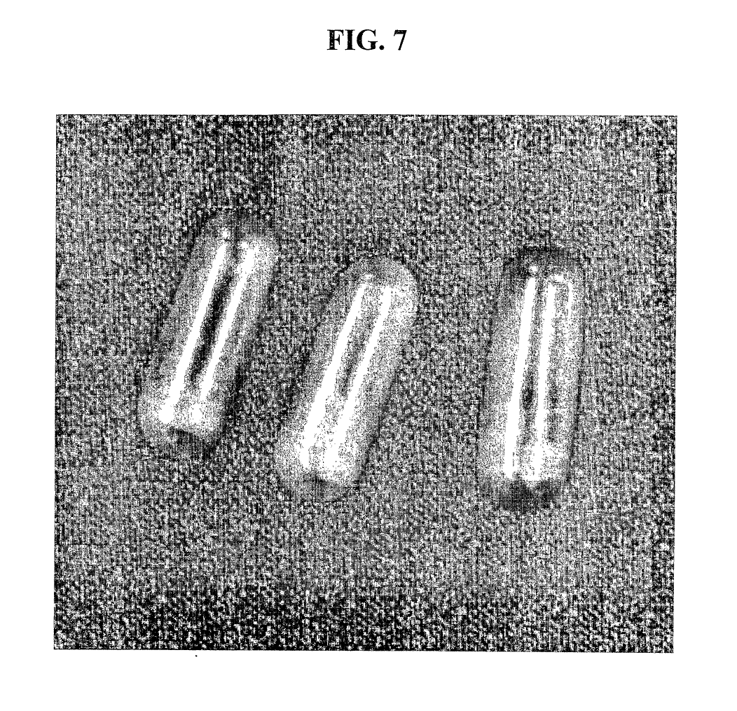

As shown in FIG. 7, the results showed that, if the amount of the waterchestnut starch was 25-55 g, it would be prepared into hard capsules even when the gelling agent was not added, similarly to the case of the mungbean starch.

### Example 7

### Examination of Characteristics of Starch

The present inventors have examined the molecular characteristics and paste viscosity characteristics of each of the starches (potato starch, mungbean starch and waterchestnut starch) used in Examples 1 to 3.

(7-1) Examination of Molecular Characteristics

The molecular characteristics of the starches were analyzed by the use of a medium-pressure system with multi-angle laser light scattering (MALLS) and refractive index (RI) detectors. The analysis results are shown in Table 12 below.

TABLE 12
Molecular characteristics of each starch

| | Total | Molecular | Molecular | |
|---|---|---|---|---|
| | molecular | weight of | weight of | Amylopectin: |
| | weight | amylopectin | amylose | amylose |
| Potato starch | 6.1 × 10⁸ | 2.6 × 10⁸ | 1.8 × 10⁸ | 89:11 |
| Mungbean starch | 9.0 × 10⁷ | 1.6 × 10⁸ | 3.9 × 10⁷ | 70:30 |
| Waterchestnut | 1.2 × 10⁷ | 1.3 × 10⁸ | 3.4 × 10⁷ | 76:24 |
| starch | | | | |

(7-2) Paste Viscosity Characteristics

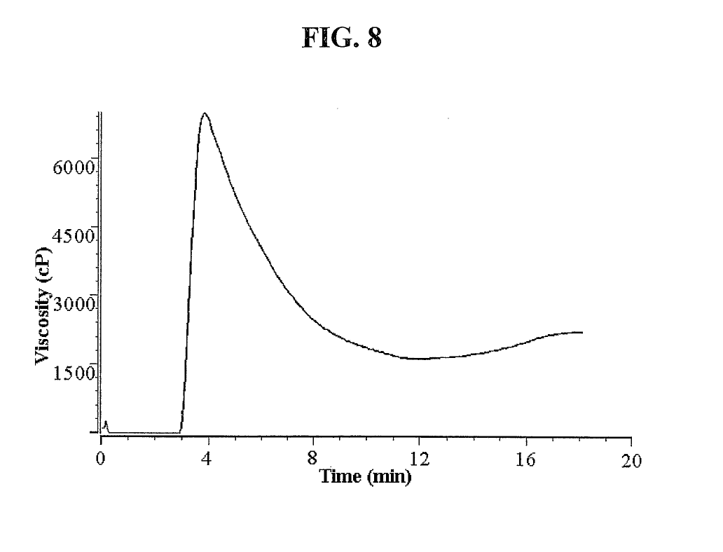

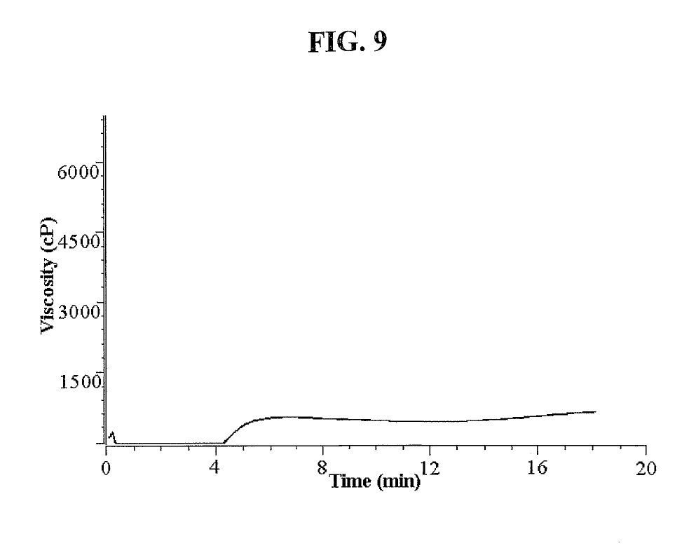

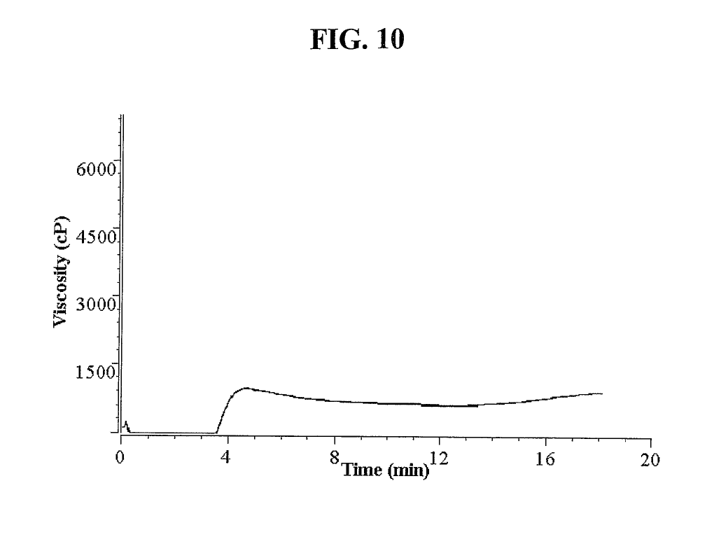

The paste viscosity of each starch was analyzed with a rapid visco analyzer (RVA) (Newport Scientific, Australia). The analysis results are given in Table 13 below. Also, the paste viscosities of each starch are graphically shown in FIGS. 8 to 10, respectively.

TABLE 13
Paste viscosity characteristics of each starch

| | pasting | Peak | Peak | | | Final |
|---|---|---|---|---|---|---|
| | temperature | temperature | viscosity | Breakdown | Setback | viscosity |
| | (° C.) | (° C.) | (cP) | (cP) | (cP) | (cP) |
| Potato starch | 65 | 75 | 6966 | 5385 | 627 | 2200 |
| Mungbean starch | 78 | 95 | 500 | 112 | 222 | 610 |
| Waterchestnut starch | 70 | 82 | 955 | 382 | 295 | 868 |

### INDUSTRIAL APPLICABILITY

As described above, the present invention provides the film-forming composition suitable for hard capsules, which comprises a small amount of starch, a plasticizer and a gelling agent. The films and hard capsules comprising the composition according to the present invention have water-soluble, biodegradable and edible properties, and at the same time, are excellent in mechanical properties. Particularly, they are made of natural plant components, and thus, advantageous in that they are harmless to the human body and do not cause environmental contamination problems. Accordingly, the inventive composition, and the hard capsules and films comprising the same, will be useful in various industrial fields, including pharmaceutical field and food field.

1. A film-forming composition for hard capsules, comprising 7-12% by weight of starch, 1-6% by weight of a plasticizer, 0.7-3% by weight of a gelling agent, and 79-91.3% by weight of water, wherein the starch comprises 10-30% by weight of amylose.

2. (canceled)

3. The composition of claim 1, wherein the starch is selected any one from the group consisting of potato starch, waterchestnut starch, and mungbean starch.

4. The composition of claim 1, wherein the plasticizer is any one selected from the group consisting of glycerol, sorbitol, polyethylene glycerol (PEG), mannitol, maltitol, and mixtures thereof.

5. The composition of claim 1, wherein the gelling agent is any one selected from the group consisting of carrageenan, agar gum, pectin, alginate, gum arabic, xanthan gum, gellan, and mixtures thereof.

6. A method for preparing a film-forming composition for hard capsules, the method comprising the steps of:
(a) adding 1-6% by weight of a plasticizer based on the total weight of the composition to 79-91.3% of water based on the total weight of the composition, and stirring the solution at 25-60° C.;
(b) adding 0.7-3% by weight of a gelling agent based on the total weight of the composition to the solution prepared in the step (a), and stirring the gelling agent-comprising solution at 50±5° C.; and
(c) adding 7-12% by weight of starch based on the total weight of the composition to the solution prepared in the step (b), wherein the starch comprises 10-30% by weight of amylose, and stirring the starch-comprising solution while gradually elevating the temperature of the solution to 90±5° C.

7. A film-forming composition for hard capsules, comprising 5-11% by weight of waterchestnut starch or mungbean starch, 1-3% by weight of a plasticizer, and 88-94% by weight of water.

8. The composition of claim 7, wherein the starch comprises 24-30% by weight of amylose.

9. The composition of claim 7, wherein the plasticizer is any one selected from the group consisting of glycerol, sorbitol, polyethylene glycerol (PEG), mannitol, maltitol, and mixtures thereof.

10. A method for preparing a film-forming composition for hard capsules, the method comprising the steps of:
(a) adding 1-3% by weight of a plasticizer based on the total weight of the composition to 88-94% by weight of water based on the total weight of the composition, and stirring the solution at 25-60° C.; and
(b) adding 5-11 % by weight of waterchestnut starch or mungbean starch based on the total weight of the composition to the solution prepared in the step (a), and stirring the starch-comprising solution while gradually elevating the temperature of the solution to 90±5° C.

11. A film for hard capsules, which comprises the composition of claim 1.

12. A hard capsule comprising the composition of claim 1.

13. A film for hard capsules, which comprises the composition of claim 7.

14. A hard capsule comprising the composition of claim 7.
